(19) United States
(12) Patent Application Publication
(10) Pub. No.: US 2026/0263477 A1
(43) Pub. Date: Sep. 10, 2026

(54) METHODS OF TREATING CANCER

(71) Applicant: Stichting Radboud universitair medisch centrum, Nijmegen (NL)

(72) Inventor: Rob ter Heine, Nijmegen (NL)

(73) Assignee: Stichting Radboud universitair medisch centrum, Nijmegen (NL)

(21) Appl. No.: 19/559,253

(22) Filed: Mar. 6, 2026

Related U.S. Application Data
(60) Provisional application No. 63/768,445, filed on Mar. 7, 2025.

(51) Int. Cl. A61K 31/5377; A61K 9/00; A61K 31/4745; A61K 31/513; A61K 31/519; A61K 31/555; A61K 39/00; A61P 35/04; C07K 16/28

(52) U.S. Cl. CPC A61K 31/5377; A61K 9/0019; A61K 9/0053; A61K 31/4745; A61K 31/513; A61K 31/519; A61K 31/555; A61P 35/04; C07K 16/2863; A61K 2039/54; A61K 2039/545

(57) ABSTRACT

Provided herein are methods of treating cancer comprising a KRAS G12C mutation in a patient who is in further need for treatment with rivaroxaban. Sotorasib is both a moderate inducer of cytochrome P450 enzyme 3A (CYP3A) and p-glycoprotein (P-gp) inhibitor. Rivaroxaban is a substrate for CYP3A and P-gp. The method of treating cancer in the patient involves co-administering sotorasib and rivaroxaban using standard doses of both sotorasib and rivaroxaban without dose adjustment with regard to the either the rivaroxaban or sotorasib, respectively.

### CROSS-REFERENCE TO RELATED APPLICATIONS

This application claims the benefit of priority to U.S. Provisional Patent Application No. 63/768,445, filed Mar. 7, 2025.

### FIELD

Provided herein are methods for treating cancer using a combination of sotorasib and rivaroxaban.

### BACKGROUND

Kirsten rat sarcoma (KRAS) G12C mutation has been identified as a putative oncogenic driver in several types of solid tumors including non-small cell lung cancer (NSCLC) (Femindez-Medarde and Santos, 2011), colorectal cancer (CRC) (Jones et al, 2017), and other solid tumors such as pancreatic, endometrial, bladder, ovarian, and small cell lung cancer (The AACR Project GENIE Consortium, 2017; Zhou et al, 2016). Sotorasib (AMG 510) is a KRAS G12C inhibitor that has shown safety and efficacy as an anti-tumor monotherapy or combination therapy. It is the active ingredient in LUMAKRAS®. The chemical name of sotorasib is 6-fluoro-7-(2-fluoro-6-hydroxyphenyl)-(1M)-1-[4-methyl-2-(propan-2-yl)pyridin-3-yl]-4-[(2S)-2-methyl-4-(prop-2-enoyl)piperazin-1-yl]pyrido[2,3-d]pyrimidin-2(1H)-one. See, e.g., LUMAKRAS® Prescribing Information Revised 01/2025. The chemical structure of sotorasib is as follows:

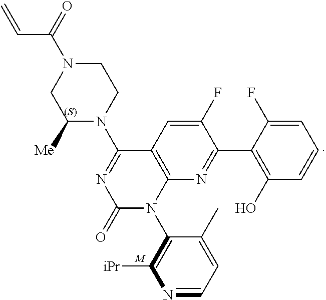

Rivaroxaban is the active ingredient in XARELTO®. See, e.g., XARELTO® Prescribing Information, Revised 02/2023. Rivaroxaban is a factor Xa inhibitor and it is used as an anticoagulant. XARELTO® is currently indicated for the following: to reduce risk of stroke and systemic embolism in nonvalvular atrial fibrillation; for treatment of deep vein thrombosis (DVT); for treatment of pulmonary embolism (PE); for reduction in the risk of recurrence of DVT or PE; for the prophylaxis of DVT, which may lead to PE in patients undergoing knee or hip replacement surgery; for prophylaxis of venous thromboembolism (VTE) in acutely ill medical patients; to reduce the risk of major cardiovascular events in patients with coronary artery disease (CAD); to reduce the risk of major thrombotic vascular events in patients with peripheral artery disease (PAD), including patients after recent lower extremity revascularization due to symptomatic PAD; for treatment of VTE and reduction in the risk of recurrent VTE in pediatric patients from birth to less than 18 years; and for thromboprophylaxis in pediatric patients 2 years and older with congenital heart disease after the Fontan procedure. The chemical name of rivaroxaban is 5-chloro-N-({(5S)-2-oxo-3-[4-(3-oxo-4-morpholinyl)phenyl]-1,3-oxazolidin-5-yl}methyl)-2-thiophenecarboxamide. The chemical structure of rivaroxaban is as follows:

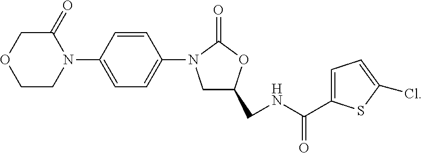 [SMILES: O=C(NC[C@H]1CN(c2ccc(N3CCOCC3=O)cc2)C(=O)O1)c1ccc(Cl)s1]

### SUMMARY

One aspect of the disclosure provides a method of treating cancer mediated by a KRAS G12C mutation in a patient in need thereof, the method comprising administering to the patient a therapeutically effective amount of sotorasib (or a pharmaceutically acceptable salt thereof) and a therapeutically effective amount of rivaroxaban. In several embodiments, the therapeutically effective amount of sotorasib (or a pharmaceutically acceptable salt thereof) is a standard dose of sotorasib (or the pharmaceutically acceptable salt thereof). In several embodiments, the therapeutically effective amount of rivaroxaban is a standard dose of rivaroxaban. In several embodiments, no clinically meaningful drug interactions are observed between rivaroxaban and sotorasib (or the pharmaceutically acceptable salt thereof) when co-administered and no dose adjustments are needed for either rivaroxaban or sotorasib (or a pharmaceutically acceptable salt thereof) with respect to each other. In several embodiments, the patient is at risk of suffering from VTE or is suffering from VTE. In several embodiments, the cancer is lung cancer, small intestine cancer, appendix cancer, colorectal cancer, endometrial cancer, pancreatic cancer, skin cancer, gastric cancer, nasal cavity cancer, or bile duct cancer. In several embodiments, the cancer is lung cancer, the lung cancer is non-small cell lung cancer (NSCLC). In several embodiments, the cancer is metastatic colorectal cancer (mCRC). In several embodiments, the cancer is locally advanced or metastatic non-small cell lung cancer (NSCLC).

Further aspects and advantages will be apparent to those of ordinary skill in the art from a review of the following detailed description. The description hereafter includes specific cases, embodiments, and examples with the understanding that the disclosure is illustrative and is not intended to limit the embodiments of the present disclosure to the specific cases, embodiments, and examples described herein.

### BRIEF DESCRIPTION OF THE DRAWINGS

FIG. 1 is a diagram showing the dosing schedule for rivaroxaban with sotorasib for patients.

FIG. 2 provides a graph of the geometric mean concentration versus time after administration of a single 20 mg dose rivaroxaban alone and with sotorasib at pharmacokinetic steady-state. The error bars represent the coefficient of variation. The y-axis is on log scale.

FIG. 3 provides a plot of geometric mean plasma concentration versus time following an oral administration of 960 mg sotorasib at pharmacokinetic steady-state in healthy subjects. The error bars represent the coefficient of variation. The y-axis is on log scale.

### DETAILED DESCRIPTION

Disclosed herein are methods of treating cancer mediated by a KRAS G12C mutation in a patient in need thereof using sotorasib while the patient is concurrently administered rivaroxaban. Rivaroxaban is often administered to cancer patients to treat or prevent, for example, VTE. Prior to the current disclosure, the administration of sotorasib and rivaroxaban was contraindicated. In the studies provided in the present disclosure, it has now been demonstrated that sotorasib (or pharmaceutically acceptable salts thereof) and rivaroxaban can be co-administered without meaningful clinical drug interactions. Thus, sotorasib (or pharmaceutically acceptable salts thereof) and rivaroxaban can be co-administered at therapeutically effective doses without dose adjustment for either sotorasib (e.g., as provided in LUMAKRAS®) or rivaroxaban (e.g., as provided in XARELTO®).

### Definitions

The following definitions are provided to assist in understanding the scope of this disclosure. Unless defined otherwise, all technical and scientific terms used herein have the same meaning as is commonly understood by one of ordinary skill in the art to which this disclosure belongs.

The term Cₚₑₐₖ refers to the highest plasma drug concentration observed during a dosing interval after steady state has been achieved, reflecting the maximum systemic exposure under chronic dosing conditions.

The term C_{trough} refers to the lowest plasma drug concentration measured immediately before the next dose once steady state has been reached, representing the minimum systemic exposure during a dosing interval at steady state.

The term “pharmaceutically acceptable salt” refers to a salt of a compound that possesses the desired pharmacological activity of the parent compound and that is not biologically or otherwise undesirable for its end use. Pharmaceutically acceptable salts include, for example, acid addition salts formed with inorganic acids (e.g., hydrochloric acid, hydrobromic acid, sulfuric acid, nitric acid, phosphoric acid) or formed with organic acids (e.g., acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid). Pharmaceutically acceptable salts also include, for example, salts formed when an acidic proton present in the parent compound either is replaced by a metal ion (e.g., an alkali metal ion, an alkaline earth ion, or an aluminum ion) or associates with an organic base (e.g., ethanolamine, diethanolamine, triethanolamine, N-methylglucamine, dicyclohexylamine). Additionally, the salts of the compounds described herein, can exist in either hydrated or anhydrous form or as solvates with other solvent molecules.

The term “therapeutically effective amount” as used herein refers to that amount of a compound disclosed herein that elicits a desired biological or medical response in a cell, a tissue, a system, or a subject.

The term “standard dose of rivaroxaban” as used herein refers to the recommended dose of rivaroxaban for a patient without dose adjustment based on co-administration with sotorasib.

The term “standard dose of sotorasib” as used herein refers to a recommended dose of sotorasib for a patient without dose adjustment based on co-administration with rivaroxaban.

The term “subject” refers to humans and other mammals. The term “mammal” as used herein includes, for example, humans, non-human primates, cattle, sheep, goats, pigs, horses, cats, dog, rabbits, rodents (e.g., rats or mice), and monkeys. The term “patient” refers to a human subject. Human subjects include neonates, infants, juveniles, adults, and geriatric subjects.

### INTRODUCTION

Sotorasib, marketed as LUMAKRAS®, is the first KRAS G12C inhibitor approved for the treatment of cancer. Currently, sotorasib is approved for use in the U.S. and EU for the treatment of adult patients with KRAS G12C-mutated locally advanced or metastatic non-small cell lung cancer (NSCLC). LUMAKRAS® is also approved for NSCLC in several other jurisdictions worldwide. Recently, sotorasib in combination with panitumumab was approved for treating KRAS G12C-mutated mCRC in patients in the U.S. Sotorasib is also in ongoing clinical trials as a part of various treatment regimens for other forms of cancers. For instance, LUMAKRAS® in combination with, panitumumab, and FOLFIRI (a combination of leucovorin calcium (i.e., folinic acid), fluorouracil, and irinotecan hydrochloride) is currently in Phase 3 clinical testing in the U.S. as a first line treatment in patients with mCRC with KRAS G12C mutation (CodeBreaK 301). A clinical study evaluating a LUMAKRAS® and platinum doublet combination is in Phase 3 clinical testing in the U.S. as a front-line therapy in patients with Stage IV or Advanced Stage IIIB/C nonsquamous NSCLCs, negative for PD-L1, and positive for a KRAS G12C mutation (CodeBreaK 202). Sotorasib and LUMAKRAS® also has demonstrated promising efficacy in KRAS G12C mutated advanced pancreatic cancer.

In addition to the damage it causes to the tissue and organs, cancer accounts for 20% of the overall incidence of VTE in people. Patients with VTE and active cancer are at higher risk of death and major bleeding than those without cancer. See, e.g., Fernandes, 2019; Weitz, 2020. Cancers most frequently associated with a high risk of VTE are hematological malignancies, lung, pancreas, stomach, bowel, gynecologic, and brain cancers. See, e.g., Timp, 2013; Mahajan, 2022; Walker, 2013. Notably, cancer driven by mutation in the KRAS gene is a risk factor for VTE. See, e.g., Abufarhaneh, 2022. Therefore, prevention and treatment of thromboembolisms is a highly relevant issue for patients with KRAS G12C cancer mutations.

Direct oral anticoagulants (DOACs) are the standard of care in the treatment and prevention of VTE and in the prevention of thromboembolic complications of atrial fibrillation. Double-blinded randomized clinical studies support the use of DOACs to treat and prevent VTEs in cancer patients. See, e.g., Raskob, 2018; Agnelli, 2020; McBane, 2020; Young, 2018; Xiang, 2023. Rivaroxaban, marketed in the U.S. as XARELTO®, is one of the most frequently used DOACs. See, e.g., Iyer, 2023; Perreault, 2020. Rivaroxaban has successfully lowered incidences of VTEs in cancer patients. Rivaroxaban, however, is susceptible to adverse pharmacokinetic drug-drug interactions.

For instance, rivaroxaban is a substrate of both cytochrome CYP3A and P-gp. As such, inhibitors or inducers of rivaroxaban's metabolism, specifically those that interact with CYP3A and the P-gp transporter, negatively impact rivaroxaban's risk-benefit profile. See., e.g., Otten, 2022. An enzyme that decreases exposure to rivaroxaban in the body increases the risk of VTEs, while mechanisms that cause higher exposure to rivaroxaban increases the risk of uncontrolled bleeding. See, e.g., Solms, 2020; Reinecke, 2020. Thus, a combination of rivaroxaban with a strong inhibitor or inducer of rivaroxaban's metabolism is not recommended. See, e.g., European Medicines Agency (EMA), 2023. Indeed, the prescribing information for XARELTO® explicitly states that prescribers should “[a]void concomitant use of XARELTO with known combined P-gp and strong CYP3A inhibitors.”

Sotorasib is both an inducer of CYP3A4 and a P-gp inhibitor. See., e.g., Otten, 2022; Cardona, 2025. Co-administration of sotorasib with midazolam, a CYP3A4 substrate, decreased exposure to midazolam by 53% in vivo. See, e.g., Otten, 2022. Combined use of sotorasib and digoxin, a P-gp substrate, increased exposure to digoxin by 21% in vivo. Because the effect of CYP3A4 induction and P-gp inhibition on rivaroxaban was unknown prior to the current disclosure, co-administration with sotorasib was contraindicated. Id. Otten et al. explained that “[t]he net effect of CYP3A4 induction and P-gp inhibition [by sotorasib] on DOACs which are both CYP3A4 and P-gp substrates[, such as rivaroxaban,] is unknown and, accordingly, co-administration is contraindicated.” Id. at 8. Similarly, the University of Liverpool's drug interaction database (found at www.cancer-druginteractions.org; see, also, e.g., www.ddimanagers.com) stated: “Coadministration has not been studied but should be avoided.”

Potential adverse events resulting from coadministration of sotorasib and rivaroxaban also are noted in the prescribing information for both LUMAKRAS® and XARELTO®. The LUMAKRAS® prescribing information states that prescribers should “[a]void coadministration with CYP3A4 substrates for which minimal concentration changes may lead to therapeutic failures of the substrate.” The LUMAKRAS® prescribing information further states: “Avoid coadministration of LUMAKRAS with P-gp substrates, for which minimal concentration changes may lead to serious toxicities.” The prescribing information for XARELTO® states: “Combined P-gp and strong CYP3A inhibitors increase exposure to rivaroxaban and may increase the risk of bleeding. Combined P-gp and strong CYP3A inducers decrease exposure to rivaroxaban and may increase the risk of thromboembolic events.” Thus, potential dangers associated with the co-administration of sotorasib with rivaroxaban (including increased risk of bleeding or increased risk of thromboembolic events) meant medical practitioners should avoid their concomitant use.

Surprisingly, as disclosed elsewhere herein, it has now been found that there is no clinically relevant interaction between sotorasib and rivaroxaban observed when these two active agents are co-administered. Disclosed herein is a clinical trial that was performed to evaluate the effect of 14 days of once-daily dosing of 960 mg sotorasib on the pharmacokinetics and safety of a single oral 20-mg dose of rivaroxaban in healthy human subjects. This clinical trial demonstrated that, unexpectedly, rivaroxaban can be safely co-administered with sotorasib in cancer patients with cancer mediated by a KRAS G12C mutation without dose adjustments for either active agent. In view of the data provided herein, medical practitioners now have a new avenue for the treatment of KRAS G12C mediated cancer in patients at risk of suffering from or suffering from blood clot-related disorders and events (e.g., VTE).

### Methods of Use

Provided herein as Embodiment 1 is a method of treating cancer mediated by a KRAS G12C mutation in a patient in need thereof, the method comprising administering to the patient a therapeutically effective amount of sotorasib, or a pharmaceutically acceptable salt thereof, and a therapeutically effective amount of rivaroxaban; wherein the therapeutically effective amount of sotorasib or a pharmaceutically acceptable salt thereof is a standard dose of sotorasib or the pharmaceutically acceptable salt thereof, wherein the therapeutically effective amount of rivaroxaban is a standard dose of rivaroxaban; and wherein no rivaroxaban dose adjustment is performed based on a drug-drug interaction (DDI) between rivaroxaban and sotorasib. In several embodiments, the dose of rivaroxaban is not adjusted over the course of treating the patient with sotorasib and rivaroxaban. In several embodiments, the dose of sotorasib is not adjusted over the course of treating the patient with sotorasib and rivaroxaban. In several embodiments, the method comprises first administering rivaroxaban to the patient on a day on which sotorasib is first administered to the patient. In several embodiments, the method comprises first administering rivaroxaban to the patient on a day that is after a day on which sotorasib is first administered to the patient. In several embodiments, the patient is treated with sotorasib at a time when the patient has not yet reached steady state pharmacokinetics with regard to rivaroxaban. In several embodiments, the standard dose of rivaroxaban provides Cₚₑₐₖ values for the patient (e.g., at steady state) that are within expected ranges for rivaroxaban. In several embodiments, the standard dose of rivaroxaban provides C_{trough} values for the patient (e.g., at steady state) that are within expected ranges for rivaroxaban. In several embodiments, the expected range of rivaroxaban Cₚₑₐₖ values for patients is about 177 ng/mL to about 361 ng/mL. In several embodiments, the expected range of rivaroxaban C_{trough} values for patients is about 9 to about 147 ng/mL. These expected Cₚₑₐₖ and C_{trough} ranges are calculated using the techniques provided in Martin et al. and consistent with guidance from the antithrombotic policy from Dutch Association of Medical Specialists guidelines. See, e.g., Gulikers et al. In several embodiments, where the patient is administered rivaroxaban prior to sotorasib, the C_{trough} of the rivaroxaban decreases by less than 40% upon introduction of the sotorasib to the rivaroxaban regimen. In several embodiments, where the patient is administered rivaroxaban prior to sotorasib, the Cₚₑₐₖ of the rivaroxaban decreases by less than 55% upon introduction of the sotorasib to the rivaroxaban regimen.

In several embodiments, the sotorasib (or a pharmaceutically acceptable salt thereof) and rivaroxaban may be administered simultaneously. In several embodiments, the sotorasib (or a pharmaceutically acceptable salt thereof) and rivaroxaban are present in separate formulations. In several embodiments, the sotorasib (or a pharmaceutically acceptable salt thereof) can be administered just followed by the rivaroxaban. In several embodiments, the rivaroxaban can be administered just followed by the sotorasib (or a pharmaceutically acceptable salt thereof). In several embodiments, simultaneous administration includes swallowing one or more tablets comprising rivaroxaban (or a suspension of rivaroxaban) and one or more tablets comprising sotorasib as part of a single process (e.g., swallowing one or more tablets comprising one active agent and immediately thereafter or substantially immediately thereafter swallowing one or more additional tablets comprising the other active agent). In several embodiments, where sotorasib (or a pharmaceutically acceptable salt thereof) is administered to the patient more than one time per day, one dose of the sotorasib (or a pharmaceutically acceptable salt thereof) and rivaroxaban may be administered at the same time. In several embodiments, the sotorasib (or a pharmaceutically acceptable salt thereof) and rivaroxaban may be administered at separate times. In several embodiments, the sotorasib and rivaroxaban are administered minutes apart. In several embodiments, the sotorasib and rivaroxaban are administered hours apart.

In several embodiments, rivaroxaban is administered to the patient to reduce risk of stroke and systemic embolism in nonvalvular atrial fibrillation, for treatment of DVT, for treatment of PE, for reduction in the risk of recurrence of DVT or PE, for the prophylaxis of DVT, which may lead to PE in patients undergoing knee or hip replacement surgery, for prophylaxis of VTE, to reduce the risk of major cardiovascular events in patients with CAD, or to reduce the risk of major thrombotic vascular events in patients with PAD. In several embodiments, rivaroxaban is administered to the patient for the prophylaxis of VTE or to treat VTE. In several embodiments, rivaroxaban is administered to the patient for the prophylaxis of VTE. In several embodiments, rivaroxaban is administered to the patient to treat VTE. In several embodiments, the pharmaceutically acceptable salt of sotorasib is a hydrochloride salt. In several embodiments, the pharmaceutically acceptable salt of sotorasib is a phosphate salt. In several embodiments, the pharmaceutically acceptable salt of sotorasib is a mesylate salt. In several embodiments, the pharmaceutically acceptable salt of sotorasib is a succinate salt.

Provided herein as Embodiment 2 is the method of Embodiment 1, wherein no dose adjustments are needed for either the rivaroxaban or the sotorasib or the pharmaceutically acceptable salt thereof. In several embodiments, if the 90% confidence intervals (CIs) of the geometric mean ratios of maximum concentration (Cₘₐₓ) and area under the curve (AUC) are between 0.70-1.43, this is considered no clinically relevant drug interaction for rivaroxaban. See, e.g., Zhu, 2022; CHMP Assessment Report for XARELTO®. In several embodiments, the 90% CIs of the geometric mean ratios of Cₘₐₓ and AUC are within 0.70-1.43. In several embodiments, the 90% CIs of the geometric mean ratios of Cₘₐₓ and AUC are within 0.89-1.12 and 0.72-0.86, respectively. In several embodiments, the 90% CI of the geometric mean ratio of Cₘₐₓ is within 0.89-1.12. In several embodiments, the 90% CI of the geometric mean ratio of AUC is within 0.72-0.86.

Provided herein as Embodiment 3 is the method of Embodiment 1 or 2, wherein the cancer is lung cancer, small intestine cancer, appendix cancer, colorectal cancer, endometrial cancer, pancreatic cancer, skin cancer, gastric cancer, nasal cavity cancer, or bile duct cancer.

Provided herein as Embodiment 4 is the method of any one of Embodiments 1-3, wherein the cancer is lung cancer.

Provided herein as Embodiment 5 is the method of Embodiment 4, wherein the lung cancer is non-small cell lung cancer (NSCLC).

Provided herein as Embodiment 6 is the method of Embodiment 4, wherein the lung cancer is Stage IV or Advanced Stage IIIB/C Nonsquamous Non-Small Cell Lung Cancers, negative for PD-L1, and positive for KRAS p.G12C.

Provided herein as Embodiment 7 is the method of any one of Embodiments 1-3, wherein the cancer is small intestine cancer.

Provided herein as Embodiment 8 is the method of any one of Embodiments 1-3, wherein the cancer is appendix cancer.

Provided herein as Embodiment 9 is the method of any one of Embodiments 1-3, wherein the cancer is colorectal cancer.

Provided herein as Embodiment 10 is the method of Embodiment 9, wherein the colorectal cancer is metastatic colorectal cancer (mCRC).

Provided herein as Embodiment 11 is the method of any one of Embodiments 1-3, wherein the cancer is endometrial cancer.

Provided herein as Embodiment 12 is the method of any one of Embodiments 1-3, wherein the cancer is pancreatic cancer.

Provided herein as Embodiment 13 is the method of any one of Embodiments 1-3, wherein the cancer is skin cancer.

Provided herein as Embodiment 14 is the method of any one of Embodiments 1-3, wherein the cancer is gastric cancer.

Provided herein as Embodiment 15 is the method of any one of Embodiments 1-3, wherein the cancer is nasal cavity cancer.

Provided herein as Embodiment 16 is the method of any one of Embodiments 1-3, wherein the cancer is bile duct cancer.

Provided herein as Embodiment 17 is the method of any one of Embodiments 1-16, comprising administering the sotorasib or the pharmaceutically acceptable salt thereof to the patient once daily.

Provided herein as Embodiment 18 is the method of any one of Embodiments 1-16, comprising administering the sotorasib or the pharmaceutically acceptable salt thereof to the patient twice daily.

Provided herein as Embodiment 19 is the method of any one of Embodiments 1-18, wherein the sotorasib or the pharmaceutically acceptable salt thereof is administered orally.

Provided herein as Embodiment 20 is the method of any one of Embodiments 1-19, wherein the sotorasib or the pharmaceutically acceptable salt thereof is administered as one or more tablets.

Provided herein as Embodiment 21 is the method of any one of Embodiments 1-20, comprising administering 240 mg, 480 mg, or 960 mg of sotorasib to the patient daily. In several embodiments, for example, the standard dose of sotorasib is 240 mg, 480 mg, or 960 mg daily. In several embodiments, the sotorasib is administered once daily (QD). In several embodiments, the sotorasib is administered twice daily (BID).

Provided herein as Embodiment 22 is the method of any one of Embodiments 1-20, comprising administering 240 mg sotorasib to the patient daily.

Provided herein as Embodiment 23 is the method of any one of Embodiments 1-20, comprising administering 480 mg sotorasib to the patient daily.

Provided herein as Embodiment 24 is the method of any one of Embodiments 1-20, comprising administering 960 mg sotorasib to the patient daily.

Provided herein as Embodiment 25 is the method of any one of Embodiments 1-24, wherein the sotorasib is administered as a free base.

Provided herein as Embodiment 26 is the method of any one of Embodiments 1-25, comprising administering 15 mg, 20 mg, or 30 mg of rivaroxaban to the patient daily. In several embodiments, for example, the standard dose of rivaroxaban is 15 mg, 20 mg, or 30 mg daily. In several embodiments, the rivaroxaban is administered once daily (QD).

Provided herein as Embodiment 27 is the method of any one of Embodiments 1-25, comprising administering 15 mg rivaroxaban to the patient daily.

Provided herein as Embodiment 28 is the method of any one of Embodiments 1-25, comprising administering 20 mg rivaroxaban to the patient daily.

Provided herein as Embodiment 29 is the method of any one of Embodiments 1-25, comprising administering 30 mg rivaroxaban to the patient daily.

Provided herein as Embodiment 30 is the method of any one of Embodiments 1-29, comprising administering the rivaroxaban to the patient once daily.

Provided herein as Embodiment 31 is the method of any one of Embodiments 1-29, comprising administering the rivaroxaban to the patient twice daily.

Provided herein as Embodiment 32 is the method of any one of Embodiments 1-31, wherein the rivaroxaban is administered orally.

Provided herein as Embodiment 33 is the method of any one of Embodiments 1-32, wherein the rivaroxaban is administered as one or more tablets.

Provided herein as Embodiment 34 is the method of any one of Embodiments 1-32, wherein the rivaroxaban is administered as an oral suspension.

Provided herein as Embodiment 35 is the method of any one of Embodiments 1-34, further comprising administering a therapeutically effective amount of at least one additional pharmaceutically active agent to the patient.

Provided herein as Embodiment 36 is the method of Embodiment 35, wherein the at least one additional pharmaceutically active agent comprises carboplatin, oxaliplatin, an anti-PD-1 inhibitor, a MEK inhibitor, an EGFR inhibitor, a TOR inhibitor, a SHP2 inhibitor, a PI3K inhibitor, an AKT inhibitor, a Top1 inhibitor, a PTPN11/SHP2 inhibitor, or a combination thereof.

Provided herein as Embodiment 37 is the method of Embodiment 36, wherein the EGFR inhibitor is an EGFR antibody.

Provided herein as Embodiment 38 is the method of Embodiment 37, wherein the EGFR antibody is panitumumab.

Provided herein as Embodiment 39 is the method of Embodiment 37 or 38, wherein the therapeutically effective amount of the EGFR antibody is 6 mg/kg.

Provided herein as Embodiment 40 is the method of any one of Embodiments 37-39, wherein the EGFR antibody is administered intravenously.

Provided herein as Embodiment 41 is the method of any one of Embodiments 37-40, wherein the EGFR antibody is administered every two weeks.

Provided herein as Embodiment 42 is the method of Embodiment 37, wherein the EGFR antibody is cetuximab.

Provided herein as Embodiment 43 is the method of any one of Embodiments 35-42, wherein the at least one additional pharmaceutically active agent comprises irinotecan, 5-fluoro-1H-pyrimidine-2,4-dione (5-FU), and leucovorin or levoleucovorin.

Provided herein as Embodiment 44 is the method of any one of Embodiments 35-41, wherein the at least one additional pharmaceutically active agent comprises oxaliplatin, 5-fluoro-1H-pyrimidine-2,4-dione (5-FU), and leucovorin or levoleucovorin.

Provided herein as Embodiment 45 is the method of Embodiment 43 or 44, comprising administering 400 mg/m² leucovorin via IV administration to the patient.

Provided herein as Embodiment 46 is the method of Embodiment 43 or 44, comprising administering 200 mg/m² levoleucovorin via IV administration to the patient.

Provided herein as Embodiment 47 is the method of any one of Embodiments 43-46, comprising administering 400 mg/m² 5-FU via IV administration to the patient.

Provided herein as Embodiment 48 is the method of any one of Embodiments 43 and 45-47, comprising administering 180 mg/m² irinotecan via IV administration to the patient.

Provided herein as Embodiment 49 is the method of Embodiment 43, comprising administering via IV administration 180 mg/m² irinotecan, 400 mg/m² leucovorin, and 400 mg/m² 5-FU to the patient every two weeks IV bolus and 2400 mg/m² 5-FU IV continuous infusion over 46-48 hours to the patient.

Provided herein as Embodiment 50 is the method of Embodiment 43, comprising administering via IV administration 180 mg/m² irinotecan, 200 mg/m² levoleucovorin, and 400 mg/m² 5-FU IV bolus and 2400 mg/m² 5-FU IV continuous infusion over 46-48 hours to the patient every two weeks.

Provided herein as Embodiment 51 is the method of any one of Embodiments 44-47, wherein the dose of oxaliplatin is 85 mg/m².

Provided herein as Embodiment 52 is the method of any one of Embodiments 44-47 and 51, wherein the oxaliplatin is administered intravenously.

Provided herein as Embodiment 53 is the method of any one of Embodiments 44-47, 51, and 52, wherein the oxaliplatin is administered every two weeks.

Provided herein as Embodiment 54 is the method of Embodiment 44, comprising administering 85 mg/m² oxaliplatin intravenously every two weeks, 200 mg/m² levoleucovorin intravenously every two weeks, and 400 mg/m² 5-FU intravenously (bolus) followed by 2400 mg/m² 5-FU intravenously over 46-48 hours as a continuous infusion every two weeks.

Provided herein as Embodiment 55 is the method of Embodiment 44, comprising administering 85 mg/m² oxaliplatin intravenously every two weeks, 400 mg/m² leucovorin intravenously every two weeks, and 400 mg/m² 5-FU intravenously (bolus) followed by 2400 mg/m² 5-FU intravenously over 46-48 hours as a continuous infusion every two weeks.

Provided herein as Embodiment 56 is the method of Embodiment 36, wherein the PTPN11/SHP2 inhibitor is a pyrimidin-4(3H)-one.

Provided herein as Embodiment 57 is the method of Embodiment 36 or 56, wherein the PTPN11/SHP2 inhibitor is:

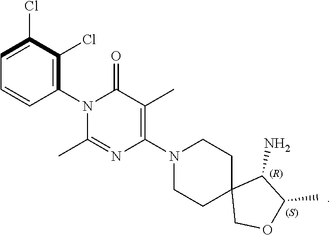

Provided herein as Embodiment 58 is the method of Embodiment 35, wherein the at least one additional pharmaceutically active agent comprises carboplatin and pemetrexed.

Provided herein as Embodiment 59 is the method of Embodiment 58, comprising administering to the subject a first regimen comprising the therapeutic amount of sotorasib, the therapeutically effective amount of carboplatin, and the therapeutically effective amount of pemetrexed to the patient.

Provided herein as Embodiment 60 is the method of Embodiment 59, wherein the first regimen is administered for a period of 4 cycles.

Provided herein as Embodiment 61 is the method of Embodiment 59 or 60, wherein each cycle is a period of 21 days.

Provided herein as Embodiment 62 is the method of Embodiment 60 or 61, wherein the carboplatin is administered on day 1 of each cycle.

Provided herein as Embodiment 63 is the method of any one of Embodiments 58-62, further comprising administering to the patient a second regimen comprising the therapeutically effective amount of sotorasib and the therapeutically effective amount of pemetrexed.

Provided herein as Embodiment 64 is the method of Embodiment 63, wherein the subject is not administered carboplatin during the second regimen.

Provided herein as Embodiment 65 is the method of any one of Embodiments 35 and 58-64, wherein the therapeutically effective amount of carboplatin is an amount corresponding to 5 mg/(mL×min) (AUC 5) multiplied with the patient's glomerular filtration rate (GFR) in mL/min increased by 25 mL/min.

Provided herein as Embodiment 66 is the method of any one of Embodiments 35 and 58-65, wherein the therapeutically effective amount of carboplatin is 750 mg or less.

Provided herein as Embodiment 67 is the method of any one of Embodiments 35 and 58-66, wherein the carboplatin is administered intravenously.

Provided herein as Embodiment 68 is the method of any one of Embodiments 58-67, wherein the therapeutically effective amount of pemetrexed is 500 mg/m².

Provided herein as Embodiment 69 is the method of any one of Embodiments 58-68, wherein pemetrexed is administered intravenously.

Provided herein as Embodiment 70 is the method of any one of Embodiments 59-69, wherein the administration of pemetrexed starts on day 1 of the first regimen.

Provided herein as Embodiment 71 is the method of any one of Embodiments 59-70, wherein the pemetrexed is administered in 21-day intervals during the first regimen and the second regimen.

### Enumerated Embodiments

The following provide exemplary illustrative enumerated embodiments not meant to limit the remainder of the disclosure.

Provided herein as Embodiment A.1 is a method of treating cancer mediated by a KRAS G12C mutation in a patient in need thereof, the method comprising administering to the patient a therapeutically effective amount of sotorasib, or a pharmaceutically acceptable salt thereof, and a therapeutically effective amount of rivaroxaban. In several embodiments, the sotorasib (or a pharmaceutically acceptable salt thereof) and rivaroxaban may be administered simultaneously. In several embodiments, the sotorasib (or a pharmaceutically acceptable salt thereof) and rivaroxaban are present in separate formulations. In several embodiments, the sotorasib (or a pharmaceutically acceptable salt thereof) can be administered just followed by the rivaroxaban. In several embodiments, the rivaroxaban can be administered just followed by the sotorasib (or a pharmaceutically acceptable salt thereof). In several embodiments, simultaneous administration includes swallowing one or more tablets comprising rivaroxaban (or a suspension of rivaroxaban) and one or more tablets comprising sotorasib as part of a single process (e.g., swallowing one or more tablets comprising one active agent and immediately thereafter or substantially immediately thereafter swallowing one or more additional tablets comprising the other active agent). In several embodiments, where sotorasib (or a pharmaceutically acceptable salt thereof) is administered to the patient more than one time per day, one dose of the sotorasib (or a pharmaceutically acceptable salt thereof) and rivaroxaban may be administered at the same time. In several embodiments, the sotorasib (or a pharmaceutically acceptable salt thereof) and rivaroxaban may be administered at separate times. In several embodiments, the sotorasib and rivaroxaban are administered minutes apart. In several embodiments, the sotorasib and rivaroxaban are administered hours apart. In several embodiments, rivaroxaban is administered to the patient to reduce risk of stroke and systemic embolism in nonvalvular atrial fibrillation, for treatment of DVT, for treatment of PE, for reduction in the risk of recurrence of DVT or PE, for the prophylaxis of DVT, which may lead to PE in patients undergoing knee or hip replacement surgery, for prophylaxis of VTE, to reduce the risk of major cardiovascular events in patients with CAD, or to reduce the risk of major thrombotic vascular events in patients with PAD. In several embodiments, rivaroxaban is administered to the patient for the prophylaxis of VTE or to treat VTE. In several embodiments, rivaroxaban is administered to the patient for the prophylaxis of VTE. In several embodiments, rivaroxaban is administered to the patient to treat VTE. In several embodiments, the pharmaceutically acceptable salt of sotorasib is a hydrochloride salt. In several embodiments, the pharmaceutically acceptable salt of sotorasib is a phosphate salt. In several embodiments, the pharmaceutically acceptable salt of sotorasib is a mesylate salt. In several embodiments, the pharmaceutically acceptable salt of sotorasib is a succinate salt.

Provided herein as Embodiment A.2 is the method of Embodiment A.1, wherein the therapeutically effective amount of sotorasib or a pharmaceutically acceptable salt thereof is a standard dose of sotorasib or the pharmaceutically acceptable salt thereof.

Provided herein as Embodiment A.3 is the method of Embodiment A.1 or A.2, wherein the therapeutically effective amount of rivaroxaban is a standard dose of rivaroxaban.

Provided herein as Embodiment A.4 is the method of any one of Embodiments A.1-A.3, wherein no clinically meaningful drug interactions are observed between the rivaroxaban and the sotorasib or the pharmaceutically acceptable salt thereof and no dose adjustments are needed for either the rivaroxaban or the sotorasib or the pharmaceutically acceptable salt thereof. In several embodiments, if the 90% confidence intervals (CIs) of the geometric mean ratios of maximum concentration (Cₘₐₓ) and area under the curve (AUC) are between 0.70-1.43, this is considered no clinically relevant drug interaction for rivaroxaban. See, e.g., Zhu, 2022; CHMP Assessment Report for XARELTO®. In several embodiments, the 90% CIs of the geometric mean ratios of Cₘₐₓ and AUC are within 0.70-1.43. In several embodiments, the 90% CIs of the geometric mean ratios of Cₘₐₓ and AUC are within 0.89-1.12 and 0.72-0.86, respectively. In several embodiments, the 90% CI of the geometric mean ratio of Cₘₐₓ is within 0.89-1.12. In several embodiments, the 90% CI of the geometric mean ratio of AUC is within 0.72-0.86.

Provided herein as Embodiment A.5 is the method of any one of Embodiments A.1-A.4, wherein the cancer is lung cancer, small intestine cancer, appendix cancer, colorectal cancer, endometrial cancer, pancreatic cancer, skin cancer, gastric cancer, nasal cavity cancer, or bile duct cancer.

Provided herein as Embodiment A.6 is the method of any one of Embodiments A.1-A.5, wherein the cancer is lung cancer.

Provided herein as Embodiment A.7 is the method of Embodiment A.5 or A.6, wherein the lung cancer is non-small cell lung cancer (NSCLC). In several embodiments, the NSCLC is locally advanced or metastatic NSCLC.

Provided herein as Embodiment A.8 is the method of any one of Embodiments A.1-A.5, wherein the cancer is small intestine cancer.

Provided herein as Embodiment A.9 is the method of any one of Embodiments A.1-A.5, wherein the cancer is appendix cancer.

Provided herein as Embodiment A.10 is the method of any one of Embodiments A.1-A.5, wherein the cancer is colorectal cancer. In several embodiments, the colorectal cancer is mCRC.

Provided herein as Embodiment A.11 is the method of any one of Embodiments A.1-A.5, wherein the cancer is endometrial cancer.

Provided herein as Embodiment A.12 is the method of any one of Embodiments A.1-A.5, wherein the cancer is pancreatic cancer.

Provided herein as Embodiment A.13 is the method of any one of Embodiments A.1-A.5, wherein the cancer is skin cancer.

Provided herein as Embodiment A.14 is the method of any one of Embodiments A.1-A.5, wherein the cancer is gastric cancer.

Provided herein as Embodiment A.15 is the method of any one of Embodiments A.1-A.5, wherein the cancer is nasal cavity cancer.

Provided herein as Embodiment A.16 is the method of any one of Embodiments A.1-A.5, wherein the cancer is bile duct cancer.

Provided herein as Embodiment A.17 is the method of any one of Embodiments A.1-A.16, comprising administering the sotorasib or the pharmaceutically acceptable salt thereof to the patient once daily.

Provided herein as Embodiment A.18 is the method of any one of Embodiments A.1-A.16, comprising administering the sotorasib or the pharmaceutically acceptable salt thereof to the patient twice daily.

Provided herein as Embodiment A.19 is the method of any one of Embodiments A.1-A.18, wherein the sotorasib or the pharmaceutically acceptable salt thereof is administered orally.

Provided herein as Embodiment A.20 is the method of any one of Embodiments A.1-A.19, wherein the sotorasib or the pharmaceutically acceptable salt thereof is administered as one or more tablets.

Provided herein as Embodiment A.21 is the method of any one of Embodiments A.1-A.20, comprising administering 240 mg, 480 mg, or 960 mg of sotorasib to the patient daily.

Provided herein as Embodiment A.22 is the method of any one of Embodiments A.1-A.20, comprising administering 240 mg sotorasib to the patient daily.

Provided herein as Embodiment A.23 is the method of any one of Embodiments A.1-A.20, comprising administering 480 mg sotorasib to the patient daily.

Provided herein as Embodiment A.24 is the method of any one of Embodiments A.1-A.20, comprising administering 960 mg sotorasib to the patient daily.

Provided herein as Embodiment A.25 is the method of any one of Embodiments A.1-A.24, wherein the sotorasib is administered as a free base.

Provided herein as Embodiment A.26 is the method of any one of Embodiments A.1-A.25, comprising administering 15 mg, 20 mg, or 30 mg of rivaroxaban to the patient daily.

Provided herein as Embodiment A.27 is the method of any one of Embodiments A.1-A.25, comprising administering 15 mg rivaroxaban to the patient daily.

Provided herein as Embodiment A.28 is the method of any one of Embodiments A.1-A.25, comprising administering 20 mg rivaroxaban to the patient daily.

Provided herein as Embodiment A.29 is the method of any one of Embodiments A.1-A.25, comprising administering 30 mg rivaroxaban to the patient daily.

Provided herein as Embodiment A.30 is the method of any one of Embodiments A.1-A.29, comprising administering the rivaroxaban to the patient once daily.

Provided herein as Embodiment A.31 is the method of any one of Embodiments A.1-A.29, comprising administering the rivaroxaban to the patient twice daily.

Provided herein as Embodiment A.32 is the method of any one of Embodiments A.1-A.31, wherein the rivaroxaban is administered orally.

Provided herein as Embodiment A.33 is the method of any one of Embodiments A.1-A.32, wherein the rivaroxaban is administered as one or more tablets.

Provided herein as Embodiment A.34 is the method of any one of Embodiments A.1-A.32, wherein the rivaroxaban is administered as an oral suspension.

Provided herein as Embodiment A.35 is the method of any one of Embodiments A.1-A.34, further comprising administering a therapeutically effective amount of at least one additional pharmaceutically active agent to the patient.

Provided herein as Embodiment A.36 is the method of Embodiment A.35, wherein the at least one additional pharmaceutically active agent comprises carboplatin, oxaliplatin, an anti-PD-1 inhibitor, a MEK inhibitor, an EGFR inhibitor, a TOR inhibitor, a SHP2 inhibitor, a PI3K inhibitor, an AKT inhibitor, a Top1 inhibitor, a PTPN11/SHP2 inhibitor, or a combination thereof.

Provided herein as Embodiment A.37 is the method of Embodiment A.36, wherein the EGFR inhibitor is an EGFR antibody.

Provided herein as Embodiment A.38 is the method of Embodiment A.37, wherein the EGFR antibody is panitumumab.

Provided herein as Embodiment A.39 is the method of any one of Embodiments A.36-A.38, wherein the therapeutically effective amount of the EGFR inhibitor is 6 mg/kg.

Provided herein as Embodiment A.40 is the method of any one of Embodiments A.36-A.39, wherein the EGFR antibody is administered intravenously.

Provided herein as Embodiment A.41 is the method of any one of Embodiments A.36-A.40, wherein the EGFR antibody is administered every two weeks.

Provided herein as Embodiment A.42 is the method of Embodiment A.37, wherein the EGFR antibody is cetuximab.

Provided herein as Embodiment A.43 is the method of any one of Embodiments A.35-A.42, wherein the at least one additional pharmaceutically active agent comprises irinotecan, 5-fluoro-1H-pyrimidine-2,4-dione (5-FU), and leucovorin or levoleucovorin.

Provided herein as Embodiment A.44 is the method of any one of Embodiments A.35-A.41, wherein the at least one additional pharmaceutically active agent comprises oxaliplatin, 5-fluoro-1H-pyrimidine-2,4-dione (5-FU), and leucovorin or levoleucovorin.

Provided herein as Embodiment A.45 is the method of Embodiment A.43 or A.44, comprising administering 400 mg/m² leucovorin via IV administration to the patient.

Provided herein as Embodiment A.46 is the method of Embodiment A.43 or A.44, comprising administering 200 mg/m² levoleucovorin via IV administration to the patient.

Provided herein as Embodiment A.47 is the method of any one of Embodiments A.43-A.46, comprising administering 400 mg/m² 5-FU via IV administration to the patient.

Provided herein as Embodiment A.48 is the method of any one of Embodiments A.43 and A.45-A.47, comprising administering 180 mg/m² irinotecan via IV administration to the patient.

Provided herein as Embodiment A.49 is the method of Embodiment A.43, comprising administering via IV administration 180 mg/m² irinotecan, 400 mg/m² leucovorin, and 400 mg/m² 5-FU to the patient every two weeks IV bolus and 2400 mg/m² 5-FU IV continuous infusion over 46-48 hours to the patient.

Provided herein as Embodiment A.50 is the method of Embodiment A.43, comprising administering via IV administration 180 mg/m² irinotecan, 200 mg/m² levoleucovorin, and 400 mg/m² 5-FU IV bolus and 2400 mg/m² 5-FU IV continuous infusion over 46-48 hours to the patient every two weeks.

Provided herein as Embodiment A.51 is the method of any one of Embodiments A.44-A.47, wherein the dose of oxaliplatin is 85 mg/m².

Provided herein as Embodiment A.52 is the method of any one of Embodiments A.44-A.47 and 51, wherein the oxaliplatin is administered intravenously.

Provided herein as Embodiment A.53 is the method of any one of Embodiments A.44-A.47, A.51, and A.52, wherein the oxaliplatin is administered every two weeks.

Provided herein as Embodiment A.54 is the method of Embodiment A.44, comprising administering 85 mg/m² oxaliplatin intravenously every two weeks, 200 mg/m² levoleucovorin intravenously every two weeks, and 400 mg/m² 5-FU intravenously (bolus) followed by 2400 mg/m² 5-FU intravenously over 46-48 hours as a continuous infusion every two weeks.

Provided herein as Embodiment A.55 is the method of Embodiment A.44, comprising administering 85 mg/m² oxaliplatin intravenously every two weeks, 400 mg/m² leucovorin intravenously every two weeks, and 400 mg/m² 5-FU intravenously (bolus) followed by 2400 mg/m² 5-FU intravenously over 46-48 hours as a continuous infusion every two weeks.

Provided herein as Embodiment A.56 is the method of Embodiment A.36, wherein the PTPN11/SHP2 inhibitor is a pyrimidin-4(3H)-one.

Provided herein as Embodiment A.57 is the method of Embodiment A.36 or A.56, wherein the PTPN11/SHP2 inhibitor is:

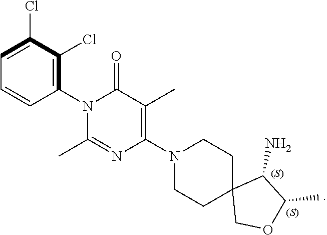

Provided herein as Embodiment A.58 is the method of Embodiment A.35, wherein the at least one additional pharmaceutically active agent comprises carboplatin and pemetrexed.

Provided herein as Embodiment A.59 is the method of Embodiment A.58, comprising administering to the subject a first regimen comprising the therapeutic amount of sotorasib, the therapeutically effective amount of carboplatin, and the therapeutically effective amount of pemetrexed to the patient.

Provided herein as Embodiment A.60 is the method of Embodiment A.59, wherein the first regimen is administered for a period of 4 cycles.

Provided herein as Embodiment A.61 is the method of Embodiment A.59 or A.60, wherein each cycle is a period of 21 days.

Provided herein as Embodiment A.62 is the method of Embodiment A.60 or A.61, wherein the carboplatin is administered on day 1 of each cycle.

Provided herein as Embodiment A.63 is the method of any one of Embodiments A.58-A.62, further comprising administering to the patient a second regimen comprising the therapeutically effective amount of sotorasib and the therapeutically effective amount of pemetrexed.

Provided herein as Embodiment A.64 is the method of Embodiment A.63, wherein the subject is not administered carboplatin during the second regimen.

Provided herein as Embodiment A.65 is the method of any one of Embodiments A.35 and A.58-A.64, wherein the therapeutically effective amount of carboplatin is an amount corresponding to 5 mg/(mL×min) (AUC 5) multiplied with the patient's glomerular filtration rate (GFR) in mL/min increased by 25 mL/min.

Provided herein as Embodiment A.66 is the method of any one of Embodiments A.35 and A.58-A.65, wherein the therapeutically effective amount of carboplatin is 750 mg or less.

Provided herein as Embodiment A.67 is the method of any one of Embodiments A.35 and A.58-A.66, wherein the carboplatin is administered intravenously.

Provided herein as Embodiment A.68 is the method of any one of Embodiments A.62-A.67, wherein the carboplatin is administered on day 1 of each cycle.

Provided herein as Embodiment A.69 is the method of any one of Embodiments A.58-A.68, wherein the therapeutically effective amount of pemetrexed is 500 mg/m².

Provided herein as Embodiment A.70 is the method of any one of Embodiments A.58-A.69, wherein pemetrexed is administered intravenously.

Provided herein as Embodiment A.71 is the method of any one of Embodiments A.59-A.70, wherein the administration of pemetrexed starts on day 1 of the first regimen.

Provided herein as Embodiment A.72 is the method of any one of Embodiments A.59-A.71, wherein the pemetrexed is administered in 21-day intervals during the first regimen and the second regimen.

The following example is given for the purpose of illustrating various embodiments of the disclosure and is not meant to limit the present disclosure in any fashion. One skilled in the art will appreciate readily that the present disclosure is well-adapted to carry out the objects and obtain the ends and advantages mentioned, as well as those objects, ends, and advantages inherent herein. Changes therein and other uses which are encompassed within the spirit of the disclosure as defined by the scope of the claims will occur to those skilled in the art.

### EXAMPLES

### Example 1: Open-Label, Single-Sequence, Pharmacokinetic Drug-Drug Interaction Study Using Sotorasib and Rivaroxaban

### SECTION 1: Study Design

This open-label, single-sequence, pharmacokinetic drug-drug interaction study (ClinicalTrial.gov (NCT06314763)) in twenty-two healthy subjects was conducted from November 2023 to March 2024 at Radboud University Medical Center, Nijmegen, the Netherlands. The protocol was approved by the local ethics committee MREC Oost-Nederland. All subjects gave informed consent before study entry.

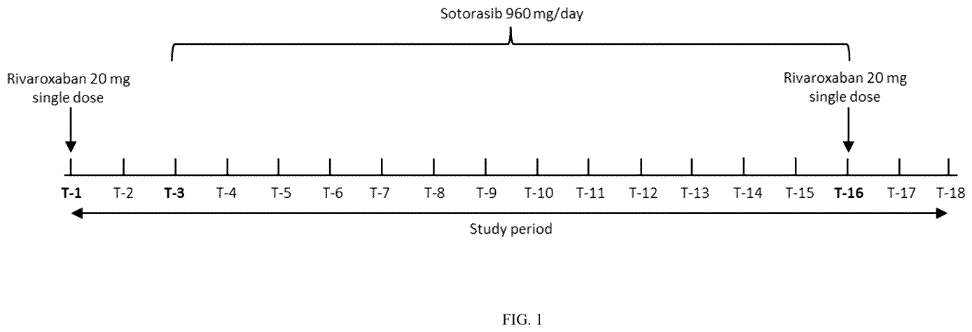

The pharmacokinetics of a single dose rivaroxaban were assessed in presence and absence of sotorasib. The study participants received rivaroxaban (20 mg single dose ingested with food) on the first study day, followed by a wash-out period of 2 days, followed by daily administration of sotorasib 960 mg once daily on day 3-16 and rivaroxaban (20 mg single dose ingested with food) on day 16 (FIG. 1).

Based on a previous pharmacokinetic drug-drug interaction study with rivaroxaban, an intra-individual coefficient of variation (CV) of 20% in AUC was assumed for sample size calculation. This CV is considered a worst-case scenario, as the observed inter-individual variation in rivaroxaban AUC and Cₘₐₓ in this previous study was approximately 240 and as intra-individual variability is assumed to be a two-fold lower than inter-individual variability. A total sample size of 20 subjects was considered sufficient to show what the 90% confidence interval of the ratio of the log-transformed AUC or Cₘₐₓ was within the bioequivalence margins of 0.8-1.25, with a power of 90% and a level of significance of 5%.

### Inclusion and Exclusion Criteria

Key inclusion criteria included age between 18 and 65 years; normal body mass index (between 18 and 30 kg/m²); not smoking more than 10 (e-)cigarettes, 2 cigars, or 2 pipes per day for at least 3 months priorto the first study day; and a good age-appropriate health condition as established by medical history and physical examination within 4 weeks prior to the first study day. Key exclusion criteria included an increased risk for bleeding or blood clotting, pregnancy, breastfeeding, and receiving concomitant drugs with a pharmacokinetic or pharmacodynamic interaction with the investigational medicinal products. The compete list of detailed eligibility criteria, is provided in Table 1.

TABLE 1
Inclusion and exclusion criteria for participants.
Inclusion criteria

| | |
|---|---|
| 1. | Subject is at least 18 and not older than 65 years at screening. |
| 2. | Subject does not smoke more than 10 (e-)cigarettes, 2 cigars, or 2 pipes per day for at least |
| | 3 months prior to the first study day. |
| 3. | Subject has a Body Mass Index of 18 to 30 kg/m2. |
| 4. | Subject is able and willing to sign the Informed Consent Form prior to screening |
| | evaluations. |
| 5. | Subject is in good age-appropriate health condition as established by medical history and |
| | physical examination within 4 weeks prior to day 1. |

Subject has a normal blood pressure and pulse rate, according to the Investigator's judgement.
Exclusion criteria

| | |
|---|---|
| 1. | An estimated glomerular filtration or creatinine clearance of less than 70 mL/min |
| 2. | Liver enzyme tests (alanine aminotransferase (ALAT), aspartate aminotransferase (ASAT), |
| | gamma-glutamyl transferase (GGT), total bilirubin, alkaline phosphatase (ALP)) greater |
| | than 2 times the upper limit of normal |
| 3. | Serum electrolytes (sodium, potassium, calcium, magnesium) outside the normal range of |
| | the NVKC reference ranges https://www.nvkc.nl/algemeen-overzicht-referentiewaarden |
| 4. | Hemocytometric values (hemoglobin, hematocrit, leukocytes, erythrocytes and |
| | thrombocytes) outside of the NVKC reference ranges https://www.nvkc.nl/algemeen- |
| | overzicht-referentiewaarden |
| 5. | A prothrombin time (PT) > 13.3 seconds |
| 6. | An activated partial thromboplastin time (APTT) >38 seconds |
| 7. | Clinically significant pathologies of the cardiovascular, bronchopulmonary, |
| | neuroendocrine systems, as well as diseases of the gastrointestinal tract, liver, |
| | kidneys and blood |
| 8. | Documented history of sensitivity/idiosyncrasy to medicinal products or excipients |
| 9. | Female subject is pregnant or lactating/breastfeeding or planning to become pregnant or |
| | breastfeed during treatment and for an additional 7 days after the last dose of sotorasib |
| 10. | Female subjects of childbearing potential unwilling to use an effective method of |
| | contraception during treatment and for an additional 7 days after the last dose of sotorasib. |
| 11. | Male subjects with a female partner of childbearing potential who are unwilling to practice |
| | sexual abstinence (refrain from heterosexual intercourse) or use contraception during |
| | treatment and for an additional 7 days after the last dose of sotorasib |
| 12. | Male subjects unwilling to abstain from donating sperm during treatment and for an |
| | additional 7 days after the last dose of sotorasib |
| 13. | Consumption of foods and beverages containing poppy seeds, St. Johns Wort, grapefruit, or |
| | Seville oranges within 7 days prior to check-in |
| 14. | Concomitant use of drugs, including herbs and food additives, with a pharmacokinetic or |
| | pharmacodynamic interaction, as assessed with most recent KNMP kennisbank and |
| | uptodate.com drug interaction databases |
| 15. | Donation of plasma or blood (450 ml or more) less than 2 months before start of the study. |
| 16. | Relevant history or current condition that might interfere with drug absorption, distribution, |
| | metabolism or excretion |
| 17. | History of or current abuse of drugs or alcohol. Alcohol abuse is defined as consuming ≥4 |
| | alcohol beverages a day. |
| 18. | Inability to understand the nature and extent of the study and the procedures required |
| 19. | Febrile illness within 3 days before Day 1 |
| 20. | History of internal bleeding of any genesis |
| 21. | Known present congenital or acquired bleeding disorders |
| 22. | History of liver disease |
| 23. | History suggestive of esophageal (including esophageal spasm, esophagitis), gastric, or |
| | duodenal ulceration or bowel disease (including but not limited to peptic ulceration, |
| | gastrointestinal bleeding, ulcerative colitis, Crohn's disease, or irritable bowel syndrome); |
| | or a history of gastrointestinal surgery other than uncomplicated appendectomy |
| 24. | Known gastrointestinal disease without active ulceration that can potentially lead to |
| | bleeding complications |
| 25. | History of vascular retinopathy |
| 26. | Known bronchiectasis |
| 27. | History of pulmonary bleeding |
| 28. | Excessive monthly vaginal bleeding |
| 29. | The following conditions known in medical history: coronary heart disease, previous |
| | cerebrovascular accident (CVA) or transient ischaemic attack |
| 30. | Positive Hepatitis B Surface Antigen (HepBsAg) (indicative of chronic Hepatitis B or |
| | recent acute hepatitis B). Negative HepBsAg with a positive for hepatitis B core antibody |
| | (Hepatitis B core antibody testing is not required for screening, however if this is done and |
| | is positive, then hepatitis B surface antibody [anti-HBs] testing is necessary. Undetectable |
| | anti-HBs in this setting would suggest unclear and possible infection and needs exclusion |
| 31. | Positive Hepatitis C virus antibody: Hepatitis C virus RNA by polymerase chain reaction |
| | (PCR) is necessary. Detectable Hepatitis C virus RNA suggests chronic hepatitis C |

### Demographics

Twenty-two healthy subjects were enrolled in this study (Table 2). One subject discontinued before study medication was taken, because of personal considerations that were unrelated to the study. Another subject had to be excluded after the first study day, because of started antibiotic treatment.

TABLE 2
Baseline characteristics of participants.

| | n = 20 |
|---|---|
| Gender, n (%) | |
| Female | 8 (40) |
| Male | 12 (60) |
| Age (y) | |
| Median (range) | 26 (19-61) |
| Baseline weight (kg) | |
| Median | 78.2 (56.7-88.6) |
| Baseline body mass index (kg/m²) | |
| Median (range) | 23.4 (19.4-29.6) |

### Blood Sampling

For every participant, blood samples were taken over a period of 48 hours to construct pharmacokinetic curves of rivaroxaban and sotorasib. Blood samples to assess rivaroxaban concentrations were collected at the following time points: t=0 (pre-dose), 0.5, 1, 1.5, 2, 3, 4, 6, 8, 12, 24, and 48 hours post dose on day 1 and day 16. Blood samples to assess sotorasib concentrations were collected at: t=0 (pre-dose), 0.5, 1, 1.5, 2, 3, 4, 6, 8, 12, and 24 hours post dose on day 16. All samples were centrifuged at 1900 G for 5 minutes at room temperature. This was done within 1.5 hours after collection. Rivaroxaban plasma samples were stored at −80° C. and sotorasib plasma samples were stored at −40° C.

### Safety and Tolerability

Safety was evaluated by monitoring treatment-emergent adverse events (AEs). AEs were graded with the most recent version (v6) of the Common Toxicity Criteria for Adverse Events (CTCAE). Incidence, severity, and relationship of AEs to the study drugs were recorded. Additional testing of liver values (ALAT, ASAT, GGT, ALP and total bilirubin) and serum electrolytes (sodium, potassium, chloride, calcium, phosphate and magnesium) were performed on the days of pharmacokinetic sampling. All AEs were followed to a satisfactory resolution or a clinically stable endpoint.

### Analysis

Concentrations of rivaroxaban and sotorasib in plasma were quantified using validated ultra-performance liquid chromatography with validated tandem mass spectrometric (UPLC-MS/MS) assays. The lower limits of quantification in these assays 2.0 μg/L and 100.0 μg/L for rivaroxaban and sotorasib, respectively.

A non-compartmental pharmacokinetic analysis was performed using Phoenix WinNonlin version 8.4 (Certara, Durham, NC, USA). To determine the area under the plasma concentration-time curve from time 0 to infinity (AUC_{0-∞}) the linear up/log down trapezoidal method was used. Additionally, Cₘₐₓ, time to maximum plasma concentration (Tₘₐₓ) and elimination half-life (T_{1/2}) were determined. Concentration values reported as below the lower limit of quantification (LLOQ) were replaced by 0.5*LLOQ for the pharmacokinetic analysis.

Geometric mean ratios and 90% confidence intervals of the test treatment (rivaroxaban and sotorasib combined) to the reference treatment (rivaroxaban alone) were determined using the method described in the European Medicines Agency (EMA) guidance on investigations of bioequivalence. To establish bioequivalence, the geometric mean ratios of AUC_{0-∞} and Cₘₐₓ should lie within the conventional acceptance range 0.80-1.25. To determine whether there is a potential, clinically relevant pharmacokinetic interaction, the following pre-defined acceptance criteria were used: if after a single dose, the 90% CIs of the geometric mean ratios of AUC_{0-∞} and Cₘₐₓ are within the range of 0.70-1.43, there is no clinically relevant pharmacokinetic interaction. The 0.70-1.43 boundaries were selected based on a previous phase III study with rivaroxaban by the license holder of rivaroxaban, assuming that a 30% change in AUC is not clinically relevant.

Additionally, a non-compartmental pharmacokinetic analysis was performed to determine the pharmacokinetic parameters of rivaroxaban pre- and postinduction.

### SECTION 2: Results and Analysis

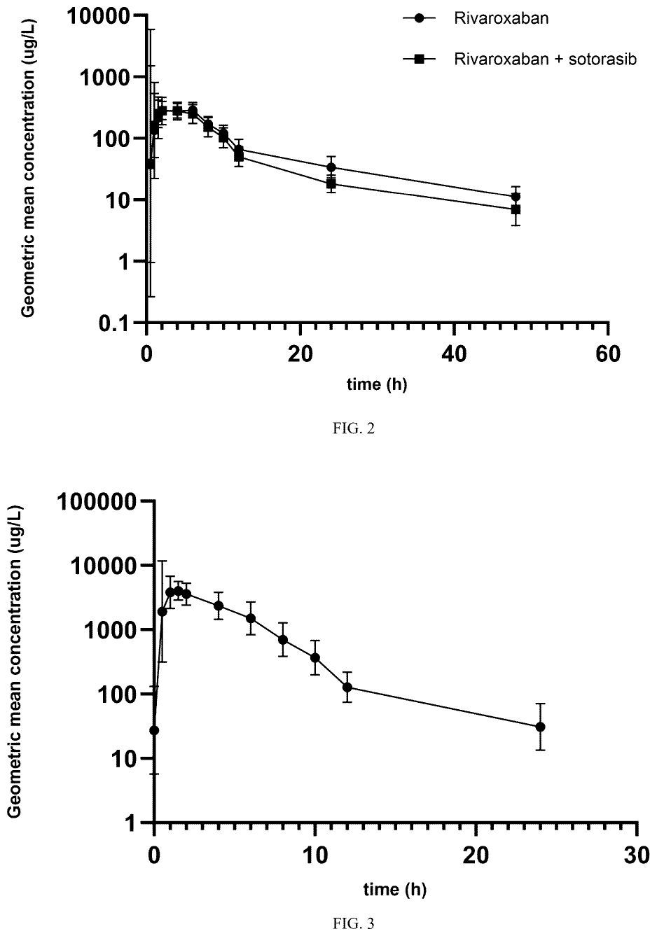

To assess the influence of sotorasib on the pharmacokinetics of rivaroxaban the geometric mean plasma concentration-time profiles of rivaroxaban pre- and postinduction were determined (FIG. 2). The test treatment (rivaroxaban and sotorasib combined) did not meet the pre-specified acceptance criteria for bioequivalence when compared to the reference treatment (rivaroxaban alone). However, it did meet the pre-specified acceptance criteria for there being no clinically relevant pharmacokinetic interaction (Table 3).

TABLE 3
Pharmacokinetic (PK) parameters of rivaroxaban in the
absence and presence of sotorasib in healthy subjects

| | | Rivaroxaban + | |
|---|---|---|---|
| | Rivaroxaban | sotorasib | |
| PK Parameter | (n = 21) | (n = 20) | Ratio (90% CI) |
| Cₘₐₓ (μg/mL) | 332 (29%) | 334 (29%) | 1.00 (0.90-1.12) |
| AUC_{0-∞} (h*μg/L) | 3,393 (27%) | 2,698 (25%) | 0.79 (0.73-0.86) |
| tₘₐₓ (h) | 2.0 (1.5-3.0) | 2.0 (1.4-3.0) | |
| t_{1/2} (h) | 14.1 (23%) | 12.8 (27%) | |
| CL/F (L/h) | 5.9 (27%) | 7.4 (25%) | |
| Vz/F (L) | 120 (37%) | 137 (38%) | |

PK parameters Cₘₐₓ, AUC_{0-∞}, T½, CL/F and Vz/F quantified as geometric means (and the percentage of geometric variation coefficient). Tₘₐₓ is shown as median plus interquartile range. The geometric mean ratios of test treatment versus reference treatment with 90% CIs are presented for Cₘₐₓ and AUC_{0-∞}.
AUC area under the curve, CI confidence interval, CL/F apparent oral clearance, Cₘₐₓ maximum concentration, PK pharmacokinetic, T½ elimination half-life, Tₘₐₓ time to maximum plasma concentration, Vz/F apparent volume of distribution.

To assess the pharmacokinetics of sotorasib the geometric mean plasma concentration-time profile was determined (FIG. 3) and a non-compartmental pharmacokinetic analysis was performed (Table 4).

TABLE 4
Pharmacokinetic (PK) parameters of sotorasib in healthy subjects.

| PK Parameter | Sotorasib (n = 20) |
|---|---|
| Cₘₐₓ (μg/mL) | 4,727 (32%) |
| AUC (h*μg/L) | 16,632 (37%) |
| tₘₐₓ (h) | 1.25 (1.0-2.0) |
| t_{1/2} (h) | 4.5 (20%) |
| CL/F (L/h) | 56.9 (37%) |
| Vz/F (L) | 369 (47%) |

This table shows the PK parameters Cₘₐₓ, AUC_{0-∞}, T½, CL/F and Vz/F as geometric means (and the percentage of geometric variation coefficient). Tₘₐₓ is shown as median plus interquartile range.
AUC area under the curve, CL/F apparent oral clearance, Cₘₐₓ maximum concentration, PK pharmacokinetic, T½ elimination half-life, Tₘₐₓ time to maximum plasma concentration, Vz/F apparent volume of distribution.

### Safety and Tolerability

There were no changes in liver (ALAT, ASAT, GGT, ALP and total bilirubin) or serum electrolytes values (sodium, potassium, chloride, calcium, phosphate and magnesium) before and after the administration of sotorasib. None of the results that were outside of the normal range were clinically significant (Table 5).

TABLE 5
Minimum and maximum liver and serum electrolytes values
before and after the administration of sotorasib.

| | |
|---|---|
| Before sotorasib | After sotorasib |

Liver values

| | | | | |
|---|---|---|---|---|
| ALAT | 12-47 | U/I | 11-42 | U/I |
| ASAT | 15-39 | U/I | 16-41 | U/I |
| Bilirubin | 4-26 | umol/L | 2-12 | umol/L |
| GGT | 7-48 | U/I | 8-52 | U/I |
| ALP | 36-116 | U/I | 32-110 | U/I |

Serum electrolytes

| | | | | |
|---|---|---|---|---|
| Sodium | 138-142 | mmol/L | 136-142 | mmol/L |
| Potassium | 3.3-4.2 | mmol/L | 3.4-4.4 | mmol/L |
| Chloride | 103-108 | mmol/L | 103-108 | mmol/L |
| Calcium | 2.23-2.55 | mmol/L | 2.14-2.44 | mmol/L |
| Magnesium | 0.73-0.92 | mmol/L | 0.72-0.96 | mmol/L |
| Phosphate | 0.58-1.45 | mmol/L | 0.65-1.49 | mmol/L |

No serious adverse events were observed and none of the drop-outs in the study were related to adverse events. In total, 51 adverse events of grade 1 and 2 were observed and reported during the study. No grade 3 or 4 adverse events were observed. The grade 2 adverse events (allergic reaction to food and dyspnea) were judged to not be related to the study medication. Commonly observed adverse events included diarrhea, headache and nausea. Among all the adverse events, 15 were deemed unrelated to the study medication, 6 were considered unlikely to be related, 12 were possibly related, and 18 were probably related (Table 6).

TABLE 6
Number of adverse events with estimated
relation to study medication

| | No. of | No. of | | Relation to |
|---|---|---|---|---|
| Term | reports | participants | Grade | study medication |
| Allergic reaction | 1 | 1 | 2 | Not related |
| (food) | | | | |
| Diarrhea | 4 | 2 | 1 | Possibly/probably |
| | | | | related |
| Dry skin | 2 | 2 | 1 | Not related |
| Dysmenorrhea | 1 | 1 | 1 | Not related |
| Dyspnea | 3 | 2 | 1/2 | Not related |
| Epistaxis | 2 | 2 | 1 | Unlikely*/possibly |
| | | | | related |
| Fatigue | 1 | 1 | 1 | Unlikely related |
| Headache | 15 | 11 | 1 | Possibly/probably |
| | | | | related |
| Laryngeal | 3 | 3 | 1 | Not related |
| inflammation | | | | |
| Nausea | 7 | 5 | 1 | Possibly/probably |
| | | | | related |
| Non-cardiac | 1 | 1 | 1 | Not related |
| chest pain | | | | |
| Polydipsia | 1 | 1 | 1 | Not related |
| Presyncope | 1 | 1 | 1 | Unlikely related |
| Pruritis | 2 | 2 | 1 | Unlikely/possibly |
| | | | | related |
| Skin discoloration | 1 | 1 | 1 | Not related |
| (hands) | | | | |
| Spasticity | 1 | 1 | 1 | Not related |
| Stomach pain | 2 | 2 | 1 | Unlikely/possibly |
| | | | | related |
| Toothache | 1 | 1 | 1 | Not related |
| Urine | 1 | 1 | 1 | Possibly related |
| discoloration | | | | |
| Vertigo | 1 | 1 | 1 | Probably related |

*Epistaxis occurred 10 days after receiving rivaroxaban and was therefore deemed unlikely to be related to the study medication.

### SECTION 3: Discussion

In this study, a clinically relevant pharmacokinetic interaction between rivaroxaban and sotorasib was not observed. The boundaries for 90% confidence intervals of the geometric mean ratios for Cₘₐₓ and AUC_{0-∞} fell inside the acceptance criteria of 0.70-1.43 for clinical relevance. The observed rivaroxaban pharmacokinetics found (Cₘₐₓ 332 μg/mL; AUC_{0-∞} 3,393 h*μg/L) is in the same order of magnitude as those observed in previous studies of rivaroxaban in healthy subjects (Cₘₐₓ 173-280 μg/mL; AUC_{0-∞} 1,612-2,425 h*μg/L). See, e.g., Moore, 2011; Brings, 2019; Kubitza, 2008; Kubitza, 2005; Kubitza, 2007; Stampfuss, 2013; Scholz, 2021. Additionally, it is comparable to the profiles seen in studied patient populations (Cₘₐₓ 270 μg/mL; AUC₂₄ 2,814 h*μg/L). See Mueck, 2014. The observed intrasubject variability was moderate for rivaroxaban, which is consistent with previous studies and the summary of product characteristics.

Rivaroxaban and sotorasib were generally well-tolerated by the healthy subjects, as all adverse events with estimated relation to study medication were of grade 1 and were considered mild in intensity. These findings align with the adverse events previously observed in single-dose trials of rivaroxaban in healthy subjects. See, e.g., Moore, 2011; Brings, 2019; Kubitza, 2008; Kubitza, 2005; Kubitza, 2007; Scholz, 2021. These findings also align with prior studies of sotorasib, given sotorasib's high selectivity for the KRAS G12C mutation, which is exclusively present in tumor cells of individuals with KRAS G12C-mutated cancer. Hong, 2020.

This study was performed in healthy subjects. It should be noted that single dose pharmacokinetic rivaroxaban studies in healthy subjects are common and the gold standard to assess the impact of drug-drug interactions with DOACs. Furthermore, a drug-drug interaction study in patients on sotorasib is not considered feasible and methodologically sound, due to high co-morbidity and polypharmacy in patients with an indication for treatment with sotorasib. Moreover, exposing vulnerable patients with an indication for treatment with sotorasib to rivaroxaban, without knowing if there is a clinically significant interaction, is unethical, as it can increase their risk of bleeding. No clinically significant pharmacokinetic interaction between rivaroxaban and sotorasib has been observed in healthy subjects and this suggests that rivaroxaban is suitable for use in patients who are treated with sotorasib. Nonetheless, the prescriber should take all concurrent medications and comorbidities into account when considering the combination of sotorasib and rivaroxaban.

### CONCLUSION

Co-administration of sotorasib with rivaroxaban does not cause a clinically relevant pharmacokinetic drug-drug interaction. Therefore, no rivaroxaban dose adjustment is warranted a priori when combined with sotorasib.

### REFERENCES

Fernández-Medarde, A., et al. Ras in Cancer and Developmental Diseases. Genes & Cancer 2011, 2(3) 344-358.
Jones, R., et al, Specific Mutations in KRAS Codon 12 are Associated with Worse Overall Survival in Patients with Advanced and Recurrent Colorectal Cancer. British Journal of Cancer 2017, 116, 923-929.
The AACR Project GENIE Consortium, 2017.
Zhou, L., et al, KRAS, BRAF, and PIK3CA Mutations, and Patient Prognosis in 126 Pancreatic Cancers: Pyrosequencing Technology and Literature Review, Med. Oncol. 2016, 33(4).
XARELTO® Prescribing information Revised 02/2023.
Fernandes, C. J., et al. Cancer-Associated Thrombosis: the When, How and Why Eur. Respir. Rev. 2019, 28(151).
Weitz, J. I., et al, Cancer Associated Thrombosis in Everyday Practice. J. Thromb. Thrombolysis. 2020, 50(2), 267-77.
Timp J.F., et al, Epidemiology of cancer-associated venous thrombosis. Blood. 2013, 122(10), 1712-23.
Mahajan, A., et al, The incidence of cancer-associated thrombosis is increasing over time. Blood Adv. 2022, 6(1), 307-20.
Walker A., et al, Incidence of venous thromboembolism in patients with cancer—a cohort study using linked United Kingdom databases. Eur J Cancer. 2013, 49(6), 1404-13.
Abufarhaneh M, et al, Association between genetic mutations and risk of venous thromboembolism in patients with solid tumor malignancies: A systematic review and meta-analysis. Thromb Res. 2022, 213, 47-56.
Raskob G., et al, Edoxaban for the Treatment of Cancer-Associated Venous Thromboembolism. N. Engl. J Med. 2018, 378(7), 615-24.
Agnelli, G., et al, Apixaban for the Treatment of Venous Thromboembolism Associated with Cancer. N. Engl. J. Med. 2020, 382(17), 1599-607.
McBane R., Apixaban and dalteparin in active malignancy-associated venous thromboembolism: The ADAM VTE trial. J Thromb Haemost. 2020,18(2), 411-21.
Young A., Comparison of an Oral Factor Xa Inhibitor With Low Molecular Weight Heparin in Patients With Cancer With Venous Thromboembolism: Results of a Randomized Trial (SELECT-D). J Clin Oncol. 2018, 36(20), 2017-23.
Xiang W., et al, Management of venous thromboembolism in patients with lung cancer: a state-of-the-art review. BMJ Open Resp. Res. 2023, 10, 1-11.
Iyer G., Trends in the Use of Oral Anticoagulants for Adults With Venous Thromboembolism in the US, 2010-2020. JAMA Netw. Open. 2023, 6(3), e234059.
Perreault, S., et al, Oral Anticoagulant Prescription Trends, Profile Use, and Determinants of Adherence in Patients with Atrial Fibrillation. Pharmacotherapy. 2020, 40(1), 40-54.
Otten, L., et al, Practical recommendations to combine small-molecule inhibitors and direct oral anticoagulants in patients with nonsmall cell lung cancer. Eur. Respir. Rev. 2022, 31(164).
Cardona P, et al, Impact of Sotorasib, a KRAS G12C Inhibitor, on the Pharmacokinetics and Therapeutic Window of Digoxin, a P-Glycoprotein Substrate. Clin Pharm. in Drug Dev. 2025, 0, 1-6.
www.cancer-druginteractions.org (sotorasib and rivaroxaban)
www.ddimanagers.com (sotorasib and rivaroxaban)
Zhu P, et al. Dosing Regimen Prediction and Confirmation With Rivaroxaban for Thromboprophylaxis in Children After the Fontan Procedure: Insights From the Phase III UNIVERSE Study. Clin. Pharm. 2022, 62(2), 220-231.
CHMP Assessment Report for XARELTO®.
Solms, A., et al, Associations between model-predicted rivaroxaban exposure and patient characteristics and efficacy and safety outcomes in the treatment of venous thromboembolism. J. Thromb. Thrombolysis. 2020, 50(1), 1-11.
Reinecke, I., Associations between model-predicted rivaroxaban exposure and patient characteristics and efficacy and safety outcomes in the prevention of venous thromboembolism. J. Thromb. Thrombolysis. 2020, 50(1), 12-9.
European Medicines Agency (EMA). Xarelto: EPAR—Product Information 2023.
www.cancer-druginteractions.org (Sotorasib entry printed 18 Jan. 2025)
Moore, K., et al, Effect of multiple doses of omeprazole on the pharmacokinetics, pharmacodynamics, and safety of a single dose of rivaroxaban. J. Cardiovasc. Pharmacol. 2011, 58(6), 581-8.
Brings, A., et al, Perpetrator effects of ciclosporin (P-glycoprotein inhibitor) and its combination with fluconazole (CYP3A inhibitor) on the pharmacokinetics of rivaroxaban in healthy volunteers. Br. J. Clin. Pharmacol. 2019, 85(7), 1528-37.
Kubitza, D., et al, Dose-escalation study of the pharmacokinetics and pharmacodynamics of rivaroxaban in healthy elderly subjects. Curr. Med. Res. Opin. 2008, 24(10), 2757-65.
Kubitza, D., et al, Safety, pharmacodynamics, and pharmacokinetics of single doses of BAY 59-7939, an oral, direct factor Xa inhibitor. Clin. Pharmacol. Ther. 2005, 78(4), 412-21.
Kubitza, D., et al, Body weight has limited influence on the safety, tolerability, pharmacokinetics, or pharmacodynamics of rivaroxaban (BAY 59-7939) in healthy subjects. J. Clin. Pharmacol. 2007, 47(2), 218-26.
Stampfuss, J., The effect of food on the absorption and pharmacokinetics of rivaroxaban. Int. J. Clin. Pharmacol. Ther. 2013, 51(7), 549-61.
Scholz, I., et al, Effects of Hypericum perforatum (St John's wort) on the pharmacokinetics and pharmacodynamics of rivaroxaban in humans. Br. J. Clin. Pharmacol. 2021, 87(3), 1466-74.
Mueck, W., Clinical pharmacokinetic and pharmacodynamic profile of rivaroxaban. Clin. Pharmacokinet. 2014, 53(1), 1-16.
ClinicalTrial.gov; NCT06314763 printed 26 Feb. 2025
Hong, D., et al, KRAS (G12C) Inhibition with Sotorasib in Advanced Solid Tumors. N. Engl. J. Med. 2020, 383(13), 1207-17.
Gulikers, J., et al, Proactive monitoring of drug-drug interactions between direct oral anticoagulants and small-molecule inhibitors in patients with non-small cell lung cancer. British J of Cancer, 2024, 131, 481-490.
Martin, K., et al, Use of the Direct Oral Anticoagulants in Obese Patients: Guidance from the SSC of the ISTH. J Thromb Haemost. 2016, 14, 308-13.
Specialisten FM. Antitrombotisch beleid: Federatie Medisch specialisten; 2020 update of Mar. 24, 2021.

1. A method of treating cancer mediated by a KRAS G12C mutation in a patient in need thereof, the method comprising administering to the patient:
a therapeutically effective amount of sotorasib, or a pharmaceutically acceptable salt thereof, and
a therapeutically effective amount of rivaroxaban;
wherein the therapeutically effective amount of sotorasib or a pharmaceutically acceptable salt thereof is a standard dose of sotorasib or the pharmaceutically acceptable salt thereof,
wherein the therapeutically effective amount of rivaroxaban is a standard dose of rivaroxaban;
wherein no rivaroxaban dose adjustment is performed based on a drug-drug interaction between rivaroxaban and sotorasib.

2. The method of claim 1, wherein no dose adjustments are needed for either the rivaroxaban or the sotorasib or the pharmaceutically acceptable salt thereof.

3. The method of claim 1, wherein the cancer is lung cancer, small intestine cancer, appendix cancer, colorectal cancer, endometrial cancer, pancreatic cancer, skin cancer, gastric cancer, nasal cavity cancer, or bile duct cancer.

4. The method of claim 1, wherein the cancer is lung cancer.

5. The method of claim 4, wherein the lung cancer is non-small cell lung cancer (NSCLC).

6. The method of claim 4, wherein the lung cancer is Stage IV or Advanced Stage IIIB/C Nonsquamous Non-Small Cell Lung Cancers, negative for PD-L1, and positive for KRAS p.G12C.

7.-8. (canceled)

9. The method of claim 1, wherein the cancer is colorectal cancer.

10. The method of claim 9, wherein the colorectal cancer is metastatic colorectal cancer (mCRC).

11.-16. (canceled)

17. The method of claim 1, comprising administering the sotorasib or the pharmaceutically acceptable salt thereof to the patient once daily.

18. The method of claim 1, comprising administering the sotorasib or the pharmaceutically acceptable salt thereof to the patient twice daily.

19. The method of claim 1, wherein the sotorasib or the pharmaceutically acceptable salt thereof is administered orally.

20. The method of claim 1, wherein the sotorasib or the pharmaceutically acceptable salt thereof is administered as one or more tablets.

21. The method of claim 1, wherein the standard dose of sotorasib is 240 mg, 480 mg, or 960 mg daily, wherein the sotorasib is a free base.

22. The method of claim 1, wherein the standard dose of sotorasib is 240 mg daily, wherein the sotorasib is a free base.

23. The method of claim 1, wherein the standard dose of sotorasib is 480 mg daily, wherein the sotorasib is a free base.

24. The method of claim 1, wherein the standard dose of sotorasib is 960 mg daily, wherein the sotorasib is a free base.

25. (canceled)

26. The method of claim 21, wherein the standard dose of rivaroxaban is 15 mg, 20 mg, or 30 mg daily.

27. The method of claim 21, wherein the standard dose of rivaroxaban is 15 mg of rivaroxaban daily.

28. The method of claim 21, wherein the standard dose of rivaroxaban is 20 mg daily.

29. The method of claim 21, wherein the standard dose of rivaroxaban is 30 mg daily.

30.-34. (canceled)

35. The method of claim 1, further comprising administering a therapeutically effective amount of at least one additional pharmaceutically active agent to the patient.

36. The method of claim 35, wherein the at least one additional pharmaceutically active agent comprises carboplatin, oxaliplatin, an anti-PD-1 inhibitor, a MEK inhibitor, an EGFR inhibitor, a TOR inhibitor, a SHP2 inhibitor, a PI3K inhibitor, an AKT inhibitor, a Top1 inhibitor, a PTPN11/SHP2 inhibitor, or a combination thereof.

37.-71. (canceled)

72. The method of claim 22, wherein the standard dose of rivaroxaban is 15 mg daily.

73. The method of claim 22, wherein the standard dose of rivaroxaban is 20 mg daily.

74. The method of claim 22, wherein the standard dose of rivaroxaban is 30 mg daily.

75. The method of claim 23, wherein the standard dose of rivaroxaban is 15 mg daily.

76. The method of claim 23, wherein the standard dose of rivaroxaban is 20 mg daily.

77. The method of claim 23, wherein the standard dose of rivaroxaban is 30 mg daily.

78. The method of claim 24, wherein the standard dose of rivaroxaban is 15 mg daily.

79. The method of claim 24, wherein the standard dose of rivaroxaban is 20 mg daily.

80. The method of claim 24, wherein the standard dose of rivaroxaban is 30 mg daily.
